(19) United States
(12) Patent Application Publication
(10) Pub. No.: US 2005/0074447 A1
(43) Pub. Date: Apr. 7, 2005

(54) Treatment for diabetic microvascular and macrovascular complications

(76) Inventors: Andreas M. Papas, Kingsport, TN (US); Konstantinos A. Papas, Jonesboro, TN (US); Klearchos K. Papas, Maple Grove, MN (US)

Correspondence Address: SUSAN F. JOHNSTON;SUSAN F. JOHNSTON, ATTORNEY AT LAW, P.O. BOX 4449, JOHNSON CITY, TN, 37602-4449, US

(21) Appl. No.: 10/956,538

(22) Filed: Oct. 1, 2004

Related U.S. Application Data
(60) Provisional application No. 60/507,826, filed on Oct. 1, 2003.

(51) Int. Cl. A61K 38/43; A61K 31/355

(52) U.S. Cl. 424/94.1; 514/458

(57) ABSTRACT

Disclosed herein is a method of preventing or retarding the progression of diabetic microvascular and macrovascular complications by chronically administering a therapeutically effective amount of gamma-tocopherol to a diabetic patient. It is further disclosed that a greater cytoprotective benefit is provided by administering a blend of gamma-tocotrienol and alpha-tocotrienol.

### CROSS REFERENCES TO RELATED APPLICATIONS

This application claims the benefit of U.S. Provisional Application Ser. No. 60/507,826 filed Oct. 1, 2003.

### FIELD OF THE INVENTION

The present invention relates to methods for controlling complications of diabetes using antioxidant therapy.

### BACKGROUND OF THE INVENTION

Diabetes is a chronic disease with no cure. Diabetes mellitus (type 1 and type 2) is primarily characterized by an impaired ability to metabolize carbohydrates and hyperglycemia. In type 1 diabetes, autoimmune destruction of pancreatic beta cells leads to a deficiency of insulin secretion and thus hyperglycemia. Insulin resistance (the lack of effect of insulin on its receptor) often precedes and is the hallmark of type 2 diabetes. However, the gradual death of the pancreatic beta cells in type 2 diabetes often causes a deficiency of insulin secretion. In both type 1 and type 2 diabetes, reactive oxygen species and reactive nitrogen species are produced in response to the diabetic state. In addition to oxidative stress injury from free radicals, there are also stress-activated signaling pathways that cause cell injury and death. The end result is a complex of injury formation at target tissues that leads to clinically significant diabetic microvascular and macrovascular complications.

Specific diabetic microvascular complications includes diabetic retinopathy, diabetic nephropathy, and diabetic neuropathy. Diabetic retinopathy is the leading cause of blindness in the working population. Diabetic nephropathy is common in type 2 diabetes and increases the risk of death one hundred fold. Diabetic neuropathy causes deleterious loss of sensation. The combination of decreased sensation and peripheral arterial insufficiency often leads to foot ulceration and eventual amputation. Further, diabetic neuropathy can cause autonomic dysfunction involving any part of the sympathetic or parasympathetic chains and produce manifestations such as diabetic gastroparesis and vomiting, severe diarrhea, or bladder dysfunction and urinary retention.

Diabetic macrovascular complications includes atherosclerosis, hypertension, coronary artery disease, cerebrovascular disease, and peripheral vascular disease. Macrovascular disease is the leading cause of death in patients with diabetes, causing seventy-five percent of the deaths in this population but only approximately thirty-five percent of deaths in patients without diabetes. Cardiovascular risk of death is increased three fold, cerebrovascular risk of death is increased two fold, and amputation risk is increase five fold. Thus, it should be appreciated that the complications of diabetes can eventually become as problematic as the primary disease of diabetes, if not more so. Unfortunately, at the time of diabetes diagnosis, the progression of long-term complications has already started in almost one fourth of diabetic patients.

The development of chronic hyperglycemia, elevated blood and tissue levels of free fatty acids (FFA), and elevated blood and tissue levels of advanced glycation end products (AGE), which is caused by both type 1 and type 2 diabetes. These conditions are known as diabetes related stresses (DRS). Researchers have found that these three diabetes related stresses have a causative link to microvascular and macrovascular diabetic complications.

The primary diabetes related stress, hyperglycemia, is the major causal factor in the development of diabetic microvascular complications and can mediate the adverse effects of those complications through multiple pathways. Normal glucose levels are considered below 110 mg/dl. Hyperglycemia is typically considered to be present when blood glucose levels are greater than 126 mg/dl. Blood glucose levels between 110 and 126 mg/dl are typically considered to indicate an impaired glucose tolerance. For purposes of the present invention, the diabetes related stress of hyperglycemia is considered to be present blood glucose levels at or above 110 mg/dl. Further, for purposes of the present invention, a patient is considered to be diabetic when he has hyperglycemia.

The second diabetes related stress is the production of advanced glycated end-products (AGEs). Reaction between the carbonyl end of glucose and the amino end of proteins forms reversible Schiff bases and then Amadori compounds. These early glycation products undergo further chemical reactions to become irreversibly cross-linked derivatives termed AGEs. AGEs are known to accumulate continuously on long-lived vessel wall proteins with aging and at a very accelerated rate in diabetes. AGEs are chemotactic to monocytes. AGE formation also increases when the local environment becomes oxidatively stressed.

Elevated levels of AGEs in blood are considered to be any levels above 14.5 U/ml. Total plasma AGEs is determined as described in Chiavelli, Fl, et al, J. Pediatr. 34, 486-491. An elevated level of AGE is considered to be present in tissues when positive immunohistochemical staining of tissue biopsy material can be demonstrated using antibodies against N-carboxymethyl-lysine (CML) or pentosidine, as detailed in N. Tanji et al, J. Am. Soc. Nephrol. 11: 1656-66, 2000.

The third diabetes related stress is the formation of elevated levels of blood and tissue free fatty acids (FFA). The free fatty acids of primary interest are palmitic acid, oleic acid, linoleic acid, and the esters thereof. Free fatty acids are increased in diabetes due to lack of insulin mediated inhibition of lypolysis in adipocytes and thus the rapid mobilization of triglycerides. There is no absolute number used for indicating an abnormally high level of free fatty acids in blood. Generally, fasting free fatty acid blood levels are greater than 500 mmol/L in diabetes, thus indicating an elevated level of free fatty acids.

It is an object of the present invention to provide a treatment for preventing or retarding the progression of diabetic microvascular and macrovascular complications. Further, it is an object of the present invention to reduce the likelihood of development of end-stage result of such diabetic complications including blindness, kidney failure, peripheral tissue damage, loss of use of extremities and limbs, coronary artery disease, stroke, amputation, and loss of sensation in extremities. It is a further object of the present invention to ameliorate the severity of diabetes itself by decreasing insulin resistance (type 2) or increasing insulin secretion (type 1).

### BRIEF SUMMARY OF THE INVENTION

The present invention includes a composition in the form of a dietary supplement and a topical formulation. The present composition is useful for preventing and retarding the progression of diabetic microvascular and macrovascular complications. The present composition includes a therapeutically effective amount of gamma-tocopherol, with the preferred embodiment including a therapeutically effective blend of gamma-tocopherol and alpha-tocopherol that provides a synergistic cytoprotective benefit against diabetic microvascular and macrovascular complications.

The present invention further includes a method of chronically administering a therapeutically the composition to a diabetic patient to prevent the onset or retard the progression of diabetic microvascular and macrovascular complications.

### DETAILED DESCRIPTION

The inventors have found that gamma-tocopherol is surprisingly cytoprotective effect on diabetic cells under the influence of diabetes related stresses, and thus, is therapeutically beneficial in the prevention of diabetic microvascular and macrovascular complications and in the retardation of the progression of diabetic microvascular and macrovascular complications. The therapeutic benefit of gamma-tocopherol against these diabetic complications is much greater than protection provided by alpha-tocopherol. However, it was unexpectedly found that the combination of gamma-tocopherol and alpha-tocopherol has significant synergistic cytoprotective benefits against the damage from diabetes and diabetic related stresses such that administration of a blend of gamma- and alpha-tocopherol is much more effective in treating or preventing diabetic complications than when gamma-tocopherol is used alone.

The compositions, formulations, and methods of the present invention are useful in treating and preventing diabetic microvascular complications including diabetic retinopathy, diabetic nephropathy, diabetic neuropathy. They are also effective in treating and preventing diabetic macrovascular complications including various forms of atherosclerosis including peripheral vascular disease, cerebral vascular disease, cardiovascular disease, and hypertension.

In the present invention, it should be understood that reference to any the tocopherols or tocotrienols (forms of vitamin E) includes the esters thereof and any combination of the non-esterified compound and its esters. Examples of suitable esters of any of the tocopherols and tocotrienols include, the succinate esters, polyethylene glycol succinate esters, acetates, nicotinates, and phosphates. D-alpha tocopheryl polyethylene glycol-100 succinate, the pegalated form of D-alpha-tocopherol known Vitamin E TPGS, is included herein as an ester of alpha-tocopherol.

The present invention includes a dietary supplement including a therapeutically effective concentration of gamma-tocopherol for use in preventing or retarding the progression of a diabetic microvascular or macrovascular complication.

The present invention further includes a dietary supplement having a synergistically effective blend of gamma-tocopherol and alpha-tocopherol. The combination gamma- and alpha- dietary supplement preferably includes a sufficiently high concentration of alpha-tocopherol in blend with the gamma-tocopherol so that a daily dose of the combination gamma- and alpha-tocopherol dietary supplement is more therapeutically effective for preventing or retarding the progression of said complication than it would have been in the absence of alpha-tocopherol. This includes a supplement having a blend of gamma- and alpha- tocopherols wherein the weight of the blend is more therapeutically effective than the same weight of gamma-tocopherol alone would be against diabetic microvascular and macrovascular complications.

The combination gamma- and alpha-tocopherol dietary supplement includes a weight ratio range of from about 100:1 to about 0.1:1 gamma-tocopherol to alpha-tocopherol. The preferred range is between about 10:1 to about 0.2:1 gamma-tocopherol to alpha-tocopherol, with a range of from about 5:1 to about 0.5:1 gamma-tocopherol to alpha-tocopherol being more preferred.

The present dietary supplement preferably further includes a therapeutically effective amount of coenzyme Q10 in blend with the gamma-tocopherol, with or without the alpha-tocopherol. Other compounds that are particularly useful for inclusion in the dietary supplement of the present invention include alpha lipoic acid, acetyl carnitine, beta- and delta-tocopherol, alpha-, beta-, gamma-, and delta-tocotrienol, beta-carotene, lutein, zeaxanthin, vitamin C, zinc, copper, selenium, n-acetylcysteine, chromium, and various combinations thereof.

A particularly preferred embodiment of the present invention is a dietary supplement for use in treating diabetic microvascular and macrovascular complications having a formulation including from about 30 to about 75 weight percent gamma-tocopherol; from about 20 to about 60 weight percent alpha-tocopherol; from about 5 to about 15 weight percent coenzyme Q10; up to about 30 weight percent alpha lipoic acid; up to about 10 weight percent acetyl carnitine; and up to about a total of 25 weight percent of other nutrients listed above. In addition to a dietary supplement, the same formulation as above is useful in providing a topical ointment for application to the skin or eye.

The inventors contemplate a preferred route of oral administration by way of an aqueous emulsion of the dietary supplement. A preferred aqueous emulsion includes about 80 to about 99 weight percent water, from about 0.5 to about 15 weight percent emulsifier, and from about 0.5 to about 15 weight percent of the combination gamma- and alpha-tocopherol dietary supplement. In addition to an aqueous emulsion for use as a dietary supplement, the aqueous emulsion can also be a topical emulsion for application to the skin or eye.

Suitable food grade emulsifiers for use in making an aqueous emulsion under the present invention include glycerin fatty acid esters (monoglycerides), acetic acid esters of monoglycerides (acetylated monoglyceride), lactic acid esters of monoglycerides (lactylated monoglyceride), citric acid esters of monoglycerides, succinic acid esters of monoglycerides, diacetyl tartaric acid esters of monoglycerides, polyglycerol esters of fatty acids (polyglycerol ester), polyglycerol polyricinoleate, sorbitan esters of fatty acids (sorbitan ester), propylene glycol esters of fatty acids, sucrose esters of fatty acids (sugar ester), sodium stearoyl-2-lactylate, calcium stearoyl-2-lactylate, lecithin, enzyme digested lecithin/enzyme treated lecithin, polysorbate 20, polysorbate 60, polysorbate 80, cetearyl alcohol, cetyl alcohol NF, olive oil PEG 7 esters, cetyl esters, behenyl alcohol, and vitamin E TPGS, with the preferred emulsifier being vitamin E TPGS. Vitamin E TPGS is a water-soluble form of natural-source vitamin E prepared by esterifying d-alpha-tocopheryl acid succinate with polyethylene glycol 1000. Vitamin E TPGS is a well known compound having a chemical formula of C₃₃O₅H₅₄(CH₂ CH₂O)ₙ, where “n” represents the number of polyethylene oxide moieties attached to the acid group of crystalline d-alpha tocopheryl acid succinate.

The dietary supplement and topical formulation of the present invention are made using methods well known in the art. The hydrophobic components are melt blended together. In the case of an aqueous emulsion, the hydrophobic melt is sufficiently mixed with water, emulsifier, and any non-hydrophobic nutrients to provide an emulsion.

A dosage form of any of the embodiments of the present invention preferably includes between about 50 mg to about 3,000 mg of gamma-tocopherol, with about 100 mg to about 1,000 mg gamma-tocopherol being more preferred.

In another aspect, the present invention includes a therapy or method comprising chronically administering a therapeutically effective amount of gamma-tocopherol to a diabetic patient in order to prevent or retard the progression of a diabetic microvascular or macrovascular complication. In a preferred embodiment, the more effective blend of gamma-tocopherol and alpha-tocopherol is administered.

The diabetic microvascular complications treatable by the present method include diabetic retinopathy, diabetic nephropathy, and diabetic neuropathy. The diabetic macrovascular complications treatable by the present method include various forms of atherosclerosis, including peripheral vascular disease, cerebral vascular disease, and cardiovascular disease.

The method of treatment is useful in preventing diabetic complications before they are exhibited in the diabetic patient. Moreover, the method of treatment is beneficially protective for patients who are “diabetic” under the above stated definition of having blood glucose levels anywhere above the normal 110 mg/dl, even if the patient's blood glucose levels have not yet reached diagnostic benchmark of 126 mg/dl for “full blown” diabetes. Thus, early initiation of the present method in a chronically administered fashion can completely prevent devastating end-stage results of diabetic complications. The present method of treatment is also very useful in retarding the progression of pre-manifested diabetic complications.

The following examples illustrate that the present invention is effective for treating diabetic microvascular and macrovascular complications in the absence of any other anti-diabetes drug therapy. However, the invention includes administration of the present dietary supplement in conjunction with an anti-diabetes drug therapy, including administering the dietary supplement in admixture with an anti-diabetes drug, as well as separately administering the present dietary supplement and an anti-diabetes drug.

The following examples are useful for illustrating the concept of the present invention and are not intended as any limitation on the invention or as a demonstration of the invention in actual practice. The examples show data from cell culture models to show the scientific rationale underlying the claimed invention.

### EXAMPLES

### Example 1

Rhesus monkey retinal endothelial cells (ATCC CRL-1780) were cultured overnight in 96 well culture plates in a humidified incubator at 37° C., under either basal (5 mM) or hyperglycemic (25 mM) conditions in the absence FFA or in with a 1 and 2 mM palmitate concentration illustrating an elevated FFA environment. At the end of the incubation period endothelial cell viability was measured with the MTS assay and results were expressed relative to the control (5 mM glucose no FFA). The results are shown in Table 1, where error bars represent standard deviations from the mean of triplicate measurements per condition. The relative viability of endothelial cells exposed to hyperglycemia and elevated FFA, as compared to the control, was about 45%. The data in Table 1 illustrates that FFA are cytotoxic to retinal endothelial cells and that their cytotoxicity is enhanced by hyperglycemia.

[image]

### Example 2

Retinal endothelial cells were cultured overnight in 96 well culture plates in a humidified incubator at 37° C., in the presence or absence of either 50 μM of alpha-tocopherol, 50 μM of gamma-tocopherol or 25 μM alpha-tocopherol plus 25 μM gamma-tocopherol when used in combination. The cells at each condition were then cultured for an additional 8 hours either in the presence or absence of 25 mM glucose and 2 mM palmitate. Alpha-tocopherol and gamma-tocopherol concentrations in each well were kept the same as those used in the pre-incubation phase. Cell viability was measured with the MTS assay. Results were normalized and values were expressed relative to basal glucose control (relative viability).

When alpha-tocopherol was used alone (50 mM) it did not seem to offer protection from exposure to hyperglycemia and elevated FFA. The relative cell viability remained at about 40 to 50%. When gamma-tocopherol was used alone (50 mM) it provided significant protection from exposure to hyperglycemia and elevated FFA in that the relative viability of cells was increased to about 80%. When 25 mM gamma-tocopherol and 25 mM alpha-tocopherol were both used, the combination provided remarkable protection. The cultured retinal endothelial cells exposed to hyperglycemic and elevated FFA conditions retained over 90% cell viability when treated with the combination of gamma-tocopherol and alpha-tocopherol. The results are shown in Table 2. Example 2 demonstrates that the combination of gamma-tocopherol and alpha-tocopherol has synergistic cytoprotective benefits in protecting endothelial cells against diabetes related stresses.

[image]

### Example 3

This example demonstrates that the cytoprotective properties shown by gamma-tocopherol and the combination of gamma-tocopherol and alpha-tocopherol in example 2 on retinal endothelial cells is also seen on rat INS-1 β-cells induced with diabetes. STZ was is a drug that induces diabetes in the INS-1 cells. STZ is thought to induce diabetes through oxidative damage. This study is valuable for illustrating the protection of alpha-tocopherol, gamma-tocopherol, and the combination of alpha-, and gamma-tocopherol against a compound that induces diabetes.

INS-1 cells were cultured overnight in 96 well culture plates in a humidified incubator at 37° C., in the presence or absence of different concentrations of alpha- or gamma-tocopherol and then in the presence of a combination of equimolar parts of gamma- and alpha-tocopherol (25 μM gamma-, 25 μM alpha-tocopherol). The cells at each condition were then cultured for an additional 24 hours either in the presence or absence of STZ (1 mM). Alpha-tocopherol and gamma-tocopherol concentrations in each well were kept the same as those used in the pre-incubation phase. Cell viability was measured with the MTS assay. Results were normalized and values were expressed relative to control (relative viability).

As shown in Table 3, exposure to STZ for 24 hours reduced viability and both alpha- and gamma-tocopherol protected from these effects in a dose dependent manner. The data in Table 3 shows that gamma-tocopherol was more effective than alpha-tocopherol in protecting INS-1 cells from STZ at all concentrations tested.

As shown in Table 4, treatment of the diabetic cells with the combination of alpha-tocopherol and gamma-tocopherol showed similarly remarkable results as those shown in Example 2.

[image]

The experiments clearly show a surprising cytoprotective effect of gamma-tocopherol, when used alone, against the cell death and apoptosis caused by diabetes-inducing compounds and against diabetes related stress-induced cellular death and apoptosis. Still further, the experiments show that the combination of gamma-tocopherol with alpha-tocopherol provides a significantly synergistic cytoprotective benefit against diabetes and the diabetes related stresses.

The invention has been described in detail with particular reference to preferred embodiments thereof, but it will be understood that variations and modifications can be effected within the spirit and scope of the invention.

1. A dietary supplement for use in preventing or retarding the progression of a diabetic microvascular or macrovascular complication, a daily dose of said dietary supplement comprising: a therapeutically effective concentration of gamma-tocopherol, an ester of gamma-tocopherol, or a combination thereof.

2. The dietary supplement according to claim 1 further comprising a sufficiently high concentration of alpha-tocopherol, an ester of alpha-tocopherol, or a combination thereof, in blend with said gamma-tocopherol so that said daily dose of said dietary supplement is more therapeutically effective for preventing or retarding the progression of said complication than it would have been in the absence of said alpha-tocopherol.

3. The dietary supplement according to claim 2 wherein the ratio of said gamma-tocopherol to said alpha-tocopherol is in a range of from about 10:1 to about 0.2:1 gamma-tocopherol to alpha-tocopherol.

4. The dietary supplement according to claim 3 wherein the ratio of said gamma-tocopherol to said alpha-tocopherol is in a range of from about 5:1 to about 0.5:1 gamma-tocopherol to alpha-tocopherol.

5. The dietary supplement according to claim 1 further comprising a sufficiently high concentration of coenzyme Q10 in blend with said gamma-tocopherol so that said daily dose of said dietary supplement is more therapeutically effective for preventing or retarding the progression of said complication than it would have been in the absence of said coenzyme Q10.

6. The dietary supplement according to claim 1 comprising
from about 30.0 to about 75.0 weight percent gamma-tocopherol, an ester of gamma-tocopherol, or a combination thereof,
from about 20.0 to about 60.0 weight percent alpha-tocopherol, an ester of alpha-tocopherol, or a combination thereof,
from about 5.0 to about 15.0 weight percent coenzyme Q10, from 0 to about 30.0 weight percent alpha lipoic acid,
from 0 to about 10.0 weight percent acetyl carnitine, and
up to 25 weight percent other nutrients selected from the group consisting of delta-tocotrienol and esters thereof, other tocotrienols and esters thereof, beta-carotene, lutein, zeaxanthin, vitamin C, zinc, copper, selenium, n-acetylcysteine, chromium, and a combination thereof, wherein the summation of said weight percentages equals a total of 100 weight percent.

7. An aqueous emulsion comprising from about 80 to about 99 weight percent water, from about 0.5 to about 15 weight percent emulsifier, and from about 0.5 to about 15 weight percent of the dietary supplement according to claim 6, wherein the summation of the weight percentages of said water, said emulsifier, and said dietary supplement equals a total of 100 weight percent.

8. An oral dosage form of the dietary supplement according to claim 1 wherein each said daily dose comprises a concentration of between about 50 to about 3,000 mg said gamma-tocopherol.

9. The dietary supplement according to claim 1 wherein said complication is diabetic retinopathy, diabetic nephropathy, diabetic neuropathy, or a combination thereof.

10. The dietary supplement according to claim 1 wherein said complication is a form of atherosclerosis including peripheral vascular disease, cerebral vascular disease, cardiovascular disease, or a combination thereof.

11. A topical formulation for use in preventing or retarding the progression of a diabetic microvascular or macrovascular complication, a daily dose of said of topical formulation comprising: a therapeutically effective concentration of gamma-tocopherol, an ester thereof, or a combination thereof.

12. The topical formulation according to claim 11 further comprising a sufficiently high concentration of alpha-tocopherol in blend with said gamma-tocopherol so that said daily dose of said topical formulation is more therapeutically effective for preventing or retarding the progression of said complication than it would have been in the absence of said alpha-tocopherol.

13. The topical formulation according to claim 12 further comprising a sufficiently high concentration of coenzyme Q10 in blend with said gamma-tocopherol and said alpha-tocopherol so that said daily dose of said topical formulation is more therapeutically effective for preventing or retarding the progression of said complication than it would have been in the absence of said coenzyme Q10.

14. A method of preventing or retarding the progression of a diabetic microvascular or macrovascular complication, said method comprising: chronically administering a therapeutically effective amount of gamma-tocopherol, an ester of gamma-tocopherol, or a combination thereof to a diabetic patient.

15. The method according to claim 14 wherein said administering step comprises chronically administering a blend of said gamma-tocopherol and alpha-tocopherol, an ester of alpha-tocopherol, or a combination thereof, said blend being more therapeutically effective than it would be in the absence of said alpha-tocopherol.

16. The method according to claim 14 wherein said method is conducted on a diabetic patient exhibiting a diabetic microvascular complication of diabetic retinopathy, diabetic nephropathy, diabetic neuropathy, or a combination thereof.

17. The method according to claim 14 wherein said method is conducted on a diabetic patient exhibiting no diabetic microvascular or macrovascular complications.

18. The method according to claim 14 wherein said method is conducted on a diabetic patient exhibiting a diabetic macrovascular complication of atherosclerosis, including peripheral vascular disease, cerebral vascular disease, cardiovascular disease, or a combination thereof.

19. The method according to claim 14 wherein said gamma-tocopherol is administered in conjunction with an anti-diabetes drug therapy.

20. The method according to claim 14 wherein said gamma-tocopherol is administered to a patient in the absence of an anti-diabetes drug therapy.

21. A method of preventing or retarding the progression of a diabetic retinopathy in a diabetic patient, said method comprising: chronically administering a therapeutically effective amount of gamma-tocopherol, an ester of gamma-tocopherol, or a combination thereof to the diabetic patient.

22. A method of preventing or retarding the progression of a diabetic nephropathy in a diabetic patient, said method comprising: chronically administering a therapeutically effective amount of gamma-tocopherol, an ester of gamma-tocopherol, or a combination thereof to the diabetic patient.

23. A method of preventing or retarding the progression of a diabetic neuropathy in a diabetic patient, said method comprising: chronically administering a therapeutically effective amount of gamma-tocopherol, an ester of gamma-tocopherol, or a combination thereof to the diabetic patient.
